(19) United States
(12) Patent Application Publication
(10) Pub. No.: US 2003/0195411 A1
(43) Pub. Date: Oct. 16, 2003

(54) Method and system for simulating the diameter enlargement of a lesion of a blood vessel

(76) Inventors: Francisco Sureda, Chatenay Malabry (FR); Vladislav Boutenko, Paris (FR); Eric Maurincomme, Chevreuse (FR)

Correspondence Address: Jay L. Chaskin, General Electric Company, 3135 Easton Turnpike - W3C, Fairfield, CT, 06431, US

(21) Appl. No.: 10/011,011

(22) Filed: Nov. 19, 2001

(30) Foreign Application Priority Data
Dec. 4, 2000 (FR) 00 15691

(51) Int. Cl. A61B 5/05; A61F 2/06

(52) U.S. Cl. 600/411; 600/420; 623/1.1

(57) ABSTRACT

The invention concerns a method and system for simulating the diameter enlargement of a lesion of a blood vessel by means of an endovascular prosthesis. A three-dimensional simulated image is visualized, showing the result of interaction between the lesion and the endovascular prosthesis after deployment of the latter. The three-dimensional simulated image visualized is the superposition of two three-dimensional images, a first three-dimensional simulated image showing the endovascular prosthesis deployed, taking into account the resistance of the lesion, and a second three-dimensional simulated image showing the enlarged lesion following the deployment of the endovascular prosthesis.

### CROSS REFERENCE TO RELATED APPLICATIONS

This application claims the benefit of a priority under 35 USC 119 to French Patent Application No. 0015691 filed Dec. 4, 2000, the entire contents of which are incorporated by reference.

### BACKGROUND OF THE INVENTION

The present invention concerns a method for simulating the diameter enlargement of a lesion of a blood vessel, particularly a stenosis, by means of an endovascular implant.

A stenosis is a narrowing of a duct, such as a patient's artery. Such a lesion can cause cardiac dysfunctions. The treatment of stenosis can be carried out by a surgical shunt (by-pass) operation, in which a conduit is placed which short-circuits the stenosis. But the shunt is a very difficult operation.

Another solution consists of operating on the stenosis by the endovascular implant technique. The endovascular implant or prosthesis is a wire mesh cylinder which is present at the start in contracted form. The intervention consists of inserting the endovascular implant in the artery and placing it at the stenosis. By means of a balloon placed in advance in the contracted endovascular implant, the endovascular implant is separated by inflating the balloon. However, the implant technique introduces a group of parameters to be taken into account, such as the material, the type of mesh and the size of the implant to be used, the deployment technique or inflation technique (balloon inflation rate, inflation pressure), the elastic recoil of the implant, etc. Those parameters have to be mastered in order to avoid problems of under-expansion or over-expansion of the implant, tear or gap between internal diameter of the artery and diameter of the implant. Failure to master the parameters can lead to poor coverage of the stenosis.

When the endovascular implant is placed, the balloon is deflated and then removed, and an opaque product of strong contrast is injected into the patient's arteries in order to form angiographic images and visualize the result of the operation. If the stenosis is not enlarged enough, the balloon is reinserted in order to inflate again. It may be necessary to insert a second implant when the first implant does not completely cover the stenosis. These correction operations are very difficult to perform and the stenosis operation is hard to reverse.

### BRIEF DESCRIPTION OF THE INVENTION

The invention is intended to avoid correction operations by anticipating the result of an endovascular prosthesis operation.

An embodiment of the invention provides operators with a tool facilitating the decision enabling them to choose the prosthesis to be used and the deployment (or inflation) technique to be employed for an effective operation synonymous with time saving.

An embodiment of the invention therefore is a method for simulating the diameter enlargement of a lesion of a blood vessel by means of an endovascular prosthesis. In an embodiment of the invention, a three-dimensional simulated image is visualized, showing the result of interaction between the lesion and the endovascular prosthesis after deployment of the latter. The simulated image is obtained by superposition of two three-dimensional images. In other words, the intervention is simulated in order to visualize the result.

The two three-dimensional images can advantageously comprise a first three-dimensional simulated image showing the endovascular prosthesis deployed, taking into account the resistance of the lesion, and a second three-dimensional simulated image showing the enlarged lesion following the deployment of the endovascular prosthesis.

In an embodiment of one method of the invention, the first three-dimensional simulated image showing the endovascular prosthesis deployed is obtained from a model of the prosthesis.

More precisely, the model of the prosthesis can be obtained from the mechanical characteristics of the prosthesis or from characteristics of the prosthesis and a three-dimensional image of the contracted prosthesis.

In an embodiment of method of the invention, the second three-dimensional simulated image showing the enlarged lesion is obtained from a model of the lesion.

In an embodiment of the invention, the model of the lesion is obtained from the composition and biomechanical properties of the blood vessels and surrounding atheromatous plaques and from a three-dimensional image of the lesion.

In an embodiment of the invention for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

In an embodiment of the invention, the model of the prosthesis is established as a function of the radial pressure and resistance forces on the mesh of the prosthesis.

In an embodiment of the invention, the model of the lesion is established, for example, by means of the finite-element method. The finite-element method is employed as a function of radial pressure forces applied on the internal walls of the blood vessels.

For example, on an effective deployment of the prosthesis in the lesion, the instantaneous state of the endovascular prosthesis and shape of the lesion are taken into account in order to simulate and visualize in three dimensions a future state of the endovascular prosthesis and of the lesion as a function of possible actions indicated by an operator.

An embodiment of the invention also provides for a system to simulate the diameter enlargement of a lesion of a blood vessel by means of an endovascular prosthesis. The system includes a computer equipped with data storage, and processing and display means for visualizing a three-dimensional simulated image showing the result of interaction between the lesion and the endovascular prosthesis after deployment of the latter. The three-dimensional simulated image can be obtained by superposition of two three-dimensional images. The computer is possibly connected to a pick-up system.

An embodiment of the invention also concerns a data storage means comprising a computer program, which enables a computer to execute: the procedure of synthesis of the model of an endovascular prosthesis and of the model of a lesion of a blood vessel in order to simulate the interaction between the lesion and the endovascular prosthesis after deployment of the latter, and the procedure of display on a screen of a three-dimensional simulated image showing the result of the interaction.

### BRIEF DESCRIPTION OF THE DRAWINGS

Other advantages and characteristics of the invention will appear on examination of a detailed description of a nonlimitative embodiment and of the attached drawings, in which:

FIG. 1 is a view of an artery containing a stenosis in which a contracted endovascular prosthesis is placed,

FIG. 2 is a view of the artery of FIG. 1 for which the endovascular prosthesis is deployed in order to enlarge the stenosis, and

FIG. 3 is a flow sheet of the different stages of a simulation process according to an embodiment of the invention.

### DETAILED DESCRIPTION OF THE INVENTION

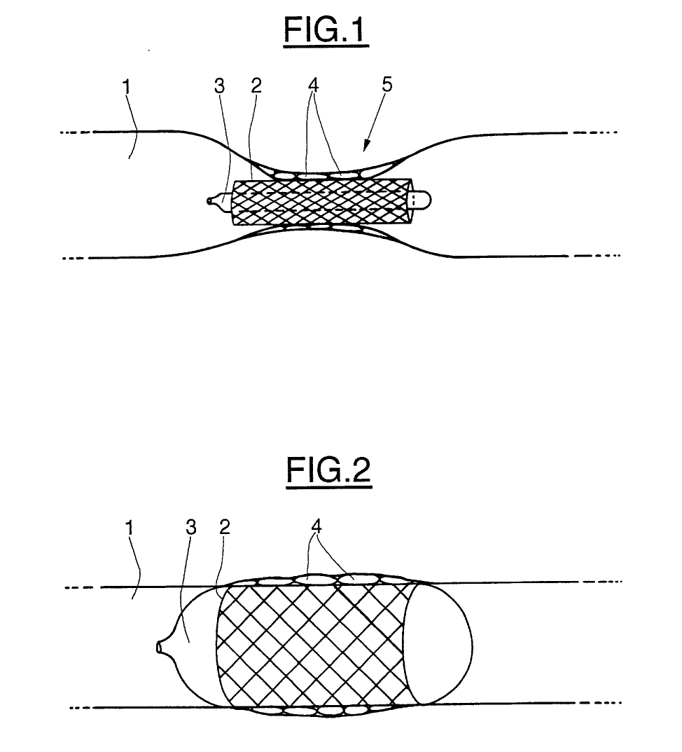

FIGS. 1 and 2 illustrate, in general, the stages of operation on a stenosis by means of an endovascular prosthesis. The artery 1 is a duct that has a narrowing 5 or a stenosis, which can prevent blood circulation. Atheromatous plaques 4, high in cholesterol, have been deposited on the internal wall of the artery 1 at the stenosis.

In the course of the text the combination of atheromatous plaques 4 and stenosis 5 will be described as lesion. To restore an artery having a constant internal diameter, an enlargement of the lesion 4, 5 is going to be made by means of an endovascular prosthesis 2. The endovascular prosthesis 2 is a deployable cylinder consisting of a wire netting (mesh). The endovascular prosthesis 2, in its contracted state, is first placed in the lesion 4, 5.

A deflated balloon is arranged inside the endovascular prosthesis 2. By means well known to the expert, the balloon 3 is inflated, which separates the mesh from the endovascular prosthesis 2.

In FIG. 2, the endovascular prosthesis 2 can be seen deployed by means of the balloon 3. The atheromatous plaques are pushed out in order to obtain an artery having a constant diameter. The stenosis is eliminated. The following stage consists of deflating the balloon and removing it from the endovascular implant which, thanks to its mechanical characteristics, remains deployed, keeping the lesion 4, 5 enlarged. However, to avoid problems of restenosis, of over- or under-deployment of the implant, of too short or too long an implant, of implant with unsuitable mechanical characteristics, etc., the method according to the invention provides for simulation of the stages described above.

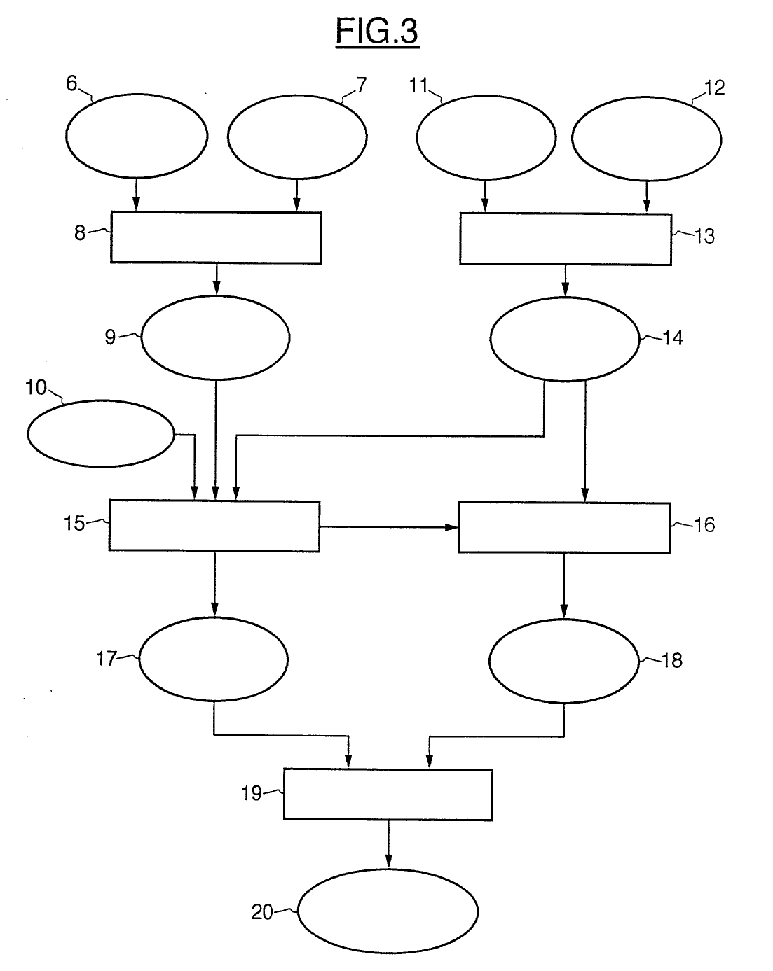

The flow chart of FIG. 3 illustrates the stages of the method according to an embodiment of the invention. A three-dimensional image 6 of the endovascular prosthesis 2 is determined in its contracted form. The mechanical characteristics 7 of the endovascular prosthesis 2 are given by the manufacturer of the endovascular prosthesis 2. The mechanical characteristics 7 concern the type of material. the form of mesh, the covering surface, the size, the curves giving the diameter as a function of pressure and the elastic recoil. The elastic recoil is a slight contraction of the endovascular prosthesis after removal of the balloon.

An equation arrangement 8 is then made to establish a parametric model 9 of the prosthesis. That parametric model describes the dynamic behavior of the prosthesis on a deployment as a function of the radial forces of pressure and resistance exerted on the mesh, and makes it possible to obtain a three-dimensional image of the prosthesis deployed for a given deployment technique, that is, balloon inflation rate and pressure.

It is also possible to consider several prostheses with a three-dimensional image 6 and specific mechanical characteristics 7 so as establish a parametric model 9 in which it is possible to choose the type of prosthesis to be used. An operator can therefore introduce different parameters in the parametric model 9, such as deployment technique and type of prosthesis. The parametric model 9 also requires other parameters coming from a parametric model 14 of the lesion, so as to take into account the forces resistant to the deployment of the prosthesis.

The parametric model 14 of the lesion is obtained by the same method as obtaining the parametric model of the prosthesis. A three-dimensional image 11 is obtained by three-dimension reconstruction from two-dimension angiographic images of the lesion. Supplementary imaging can also be used, such as echography or intravascular MRI or other imaging techniques, endovascular or not (MRI, etc.), in order to determine the composition of the lesion: composition of the artery, composition of the atheromatous plaques and composition of elements surrounding the artery at the stenosis.

The biomechanical properties 12 of the lesion concern, in particular, the artery and the atheromatous plaques. The type of artery membrane, the diameter of the artery, the form of atheromatous plaques, the gravity of the stenosis and the composition of the atheromatous plaques are distinguished in the biomechanical properties and can be determined by intravascular echography or other imaging techniques, endovascular or not (MRI, etc.). The equation arrangement of the three-dimensional image 11 of the lesion and biomechanical properties 12 of the lesion makes it possible to establish a parametric model 14 of the lesion describing the behavior of the lesions when radial pressure forces are applied on the internal walls of the artery. The behavior can be used, for example, by means of algorithms of finite elements; see the article “Echographie endocoronaire et angioplastie” [Endocoronary echography and angioplasty] (G. Finet), published in ARCHIVES DES MALADIES DU COEUR ET DES VAISSEAUX [Archives of Heart and Vascular Diseases], Volume 92, No. 11, November 1999. The parametric model 14 of the lesion makes it possible to determine a resultant three-dimensional image of the execution of the parametric model 14 for initial parameters such as radial pressure forces.

Thus, for a given lesion, a parametric model 14 is determined; for a given prosthesis, a parametric model 9 is determined; and a given technique (pressure, rate of inflation) is defined by operating parameters 10 which are pressure and inflation rate values. In stage 15, the two parametric models 9 and 14 are executed. The operating parameters 10 are introduced in the parametric model 9 to determine the radial pressure forces exerted by the implant on the internal walls of the artery. And, to determine the final state of the prosthesis deployed, it is necessary to take into account the resistance forces due to atheromatous plaques. The radial pressure forces, obtained from the operating parameters, are introduced in the parametric model 14 to determine the resistant forces.

The resistant forces are then used by the parametric model 9 of the prosthesis to determine a three-dimensional image 17 of the prosthesis deployed.

After execution of the parametric models 9 and 14 in stage 15, the radial pressure forces, the radial resistance forces and the final state of the prosthesis deployed are known. A shaping 16 can then be carried out, which consists of executing the parametric model 14, parameterized by means of the radial pressure forces, radial resistance forces and final state of the prosthesis deployed, in order to determine the three-dimensional image 18 of the enlarged lesion.

The two three-dimensional images 18 and 17 are superposed in stage 19, being established on the same scale, in order to obtain a three-dimensional image 20 comprising the deployed prosthesis and the enlarged lesion. The effectiveness of the inflation technique (inflation rate and pressure) employed and of the prosthesis chosen can then be judged. When the result obtained is not satisfactory, it is then sufficient to apply the method according to the invention again by modifying the initial parameters such as inflation technique and type of prosthesis.

In addition, it is also possible in the course of an operation, in order to make sure of the result of the operation, to determine the present stage of the parameters necessary for execution of the method according to the invention, to execute the method and to visualize the simulated final state of the operation.

In practice, the method according to the invention is managed by means of software memorized in a computer containing all the means necessary for execution of a computer program, for data storage and for communication with peripheral elements.

The computer is connected to image acquisition systems and is capable of executing three-dimensional image reconstruction algorithms.

The invention thus described is therefore a simulation of an operation making possible the selection of the proper prosthesis, which reduces the time and cost of operation. Performing several simulations with different techniques improves the safety of the operation.

Various modifications in structure and/or steps and/or function may be made by one skilled in the art without departing from the scope and extent of the invention as recited in the claims.

What is claimed is:

1. Method for simulating the diameter enlargement of a lesion of a blood vessel by means of an endovascular prosthesis, wherein a three-dimensional simulated image is visualized, showing the result of interaction between the lesion and the endovascular prosthesis after deployment of the latter, obtained by superposition of two three-dimensional images.

2. Method according to claim 1, wherein the two three-dimensional images comprise a first three-dimensional simulated image showing the endovascular prosthesis deployed, taking into account the resistance of the lesion, and a second three-dimensional simulated image showing the enlarged lesion following the deployment of the endovascular prosthesis.

3. Method according to claim 2, wherein the first three-dimensional simulated image showing the endovascular prosthesis deployed is obtained from a model of the implant.

4. Method according to claim 3, wherein the model of the implant is obtained from the mechanical characteristics of the prosthesis or from characteristics of the prosthesis and a three-dimensional image of the contracted prosthesis.

5. Method according to one of claim 2, wherein the second three-dimensional simulated image showing the enlarged lesion is obtained from a model of the lesion.

6. Method according to one of claim 3, wherein the second three-dimensional simulated image showing the enlarged lesion is obtained from a model of the lesion.

7. Method according to one of claim 4, wherein the second three-dimensional simulated image showing the enlarged lesion is obtained from a model of the lesion.

8. Method according to claim 2, wherein the model of the lesion is obtained from the composition and biomechanical properties of the blood vessels and surrounding atheromatous plaques and from a three-dimensional image of the lesion.

9. Method according to claim 3, wherein the model of the lesion is obtained from the composition and biomechanical properties of the blood vessels and surrounding atheromatous plaques and from a three-dimensional image of the lesion.

10. Method according to claim 4, wherein the model of the lesion is obtained from the composition and biomechanical properties of the blood vessels and surrounding atheromatous plaques and from a three-dimensional image of the lesion.

11. Method according to claim 5, wherein the model of the lesion is obtained from the composition and biomechanical properties of the blood vessels and surrounding atheromatous plaques and from a three-dimensional image of the lesion.

12. Method according to claim 3, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

13. Method according to claim 4, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

14. Method according to claim 5, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

15. Method according to claim 6, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

16. Method according to claim 7, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

17. Method according to claim 8, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

18. Method according to claim 9, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

19. Method according to claim 10, wherein, for particular parameters concerning the deployment technique, the lesion and the vascular prosthesis, the biomechanical properties of the lesion are taken into account to execute the model of the prosthesis in order to obtain a three-dimensional image of the prosthesis deployed, and then to execute the model of the lesion in order to obtain a three-dimensional image of the enlarged lesion.

20. Method according to claim 3, wherein the model of the prosthesis is established as a function of the radial pressure and resistance forces on the mesh of the prosthesis.

21. Method according to claim 4, wherein the model of the prosthesis is established as a function of the radial pressure and resistance forces on the mesh of the prosthesis.

22. Method according to claim 5, wherein the model of the prosthesis is established as a function of the radial pressure and resistance forces on the mesh of the prosthesis.

23. Method according to claim 6, wherein the model of the prosthesis is established as a function of the radial pressure and resistance forces on the mesh of the prosthesis.

24. Method according to claim 7, wherein the model of the prosthesis is established as a function of the radial pressure and resistance forces on the mesh of the prosthesis.

25. Method according to claim 8, wherein the model of the prosthesis is established as a function of the radial pressure and resistance forces on the mesh of the prosthesis.

26. Method according to claim 9, wherein the model of the prosthesis is established as a function of the radial pressure and resistance forces on the mesh of the prosthesis.

27. Method according to claim 10, wherein the model of the prosthesis is established as a function of the radial pressure and resistance forces on the mesh of the prosthesis.

28. Method according to claim 11, wherein the model of the prosthesis is established as a function of the radial pressure and resistance forces on the mesh of the prosthesis.

29. Method according to claim 12, wherein the model of the prosthesis is established as a function of the radial pressure and resistance forces on the mesh of the prosthesis.

30. Method according to claim 13, wherein the model of the prosthesis is established as a function of the radial pressure and resistance forces on the mesh of the prosthesis.

31. Method according to claim 14, wherein the model of the prosthesis is established as a function of the radial pressure and resistance forces on the mesh of the prosthesis.

32. Method according to claim 15, wherein the model of the prosthesis is established as a function of the radial pressure and resistance forces on the mesh of the prosthesis.

33. Method according to claim 16, wherein the model of the prosthesis is established as a function of the radial pressure and resistance forces on the mesh of the prosthesis.

34. Method according to claim 17, wherein the model of the prosthesis is established as a function of the radial pressure and resistance forces on the mesh of the prosthesis.

35. Method according to claim 18, wherein the model of the prosthesis is established as a function of the radial pressure and resistance forces on the mesh of the prosthesis.

36. Method according to claim 5, wherein the model of the lesion is established by means of the finite-element method.

37. Method according to claim 6, wherein the model of the lesion is established by means of the finite-element method.

38. Method according to claim 7, wherein the model of the lesion is established by means of the finite-element method.

39. Method according to claim 8, wherein the model of the lesion is established by means of the finite-element method.

40. Method according to claim 9, wherein the model of the lesion is established by means of the finite-element method.

41. Method according to claim 10, wherein the model of the lesion is established by means of the finite-element method.

42. Method according to claim 11, wherein the model of the lesion is established by means of the finite-element method.

43. Method according to claim 12, wherein the model of the lesion is established by means of the finite-element method.

44. Method according to claim 13, wherein the model of the lesion is established by means of the finite-element method.

45. Method according to claim 14, wherein the model of the lesion is established by means of the finite-element method.

46. Method according to claim 15, wherein the model of the lesion is established by means of the finite-element method.

47. Method according to claim 16, wherein the model of the lesion is established by means of the finite-element method.

48. Method according to claim 17, wherein the model of the lesion is established by means of the finite-element method.

49. Method according to claim 18, wherein the model of the lesion is established by means of the finite-element method.

50. Method according to claim 19, wherein the model of the lesion is established by means of the finite-element method.

51. Method according to claim 20, wherein the model of the lesion is established by means of the finite-element method.

52. Method according to claim 21, wherein the model of the lesion is established by means of the finite-element method.

53. Method according to claim 22, wherein the model of the lesion is established by means of the finite-element method.

54. Method according to claim 23, wherein the model of the lesion is established by means of the finite-element method.

55. Method according to claim 24, wherein the model of the lesion is established by means of the finite-element method.

56. Method according to claim 1, wherein on an effective deployment of the prosthesis in the lesion, the instantaneous state of the endovascular prosthesis and shape of the lesion are taken into account in order to simulate and visualize in three dimensions a future state of the endovascular prosthesis and of the lesion as a function of possible actions indicated by an operator.

57. System to simulate the diameter enlargement of a lesion of a blood vessel comprising:
means for providing an endovascular prosthesis;
means for providing a computer equipped with data storage;
means for processing and display;
means for visualizing a three-dimensional simulated image showing the result of interaction between the lesion and the endovascular prosthesis after deployment of the prosthesis, the three-dimensional simulated image being obtained by superposition of two three-dimensional images; and
the means for providing a computer being optionally connected to a means a pick-up system.

58. A computer data storage means comprising a computer program, which enables a computer to execute:
the procedure of synthesis of the model of an endovascular prosthesis and of the model of a lesion of a blood vessel in order to simulate the interaction between the lesion and the endovascular prosthesis after deployment of the latter, and
the procedure of display on a screen of a three-dimensional simulated image showing the result of the interaction.
